(19) United States
(12) Patent Application Publication
(10) Pub. No.: US 2022/0037012 A1
(43) Pub. Date: Feb. 3, 2022

(54) SERVER-INITIATED TRANSMISSION OF MESSAGES TO MEDICAL DEVICES

(71) Applicant: ICU Medical, Inc., San Clemente, CA (US)

(72) Inventors: Marshall E. Fryman, Libertyville, IL (US); Matteo D. Picinich, Temecula, CA (US); Jeffrey E. Rinda, San Diego, CA (US); Glen R. Cook, Murrieta, CA (US); Yun Shao Tsai, San Diego, CA (US); Anandaraman Vithyananthan, Chennai (IN); Syedjavid Syed Khadar, Tirupur (IN); Pritish Jain, Chennai (IN); Ujjawal Kumar, Gurugram (IN)

(21) Appl. No.: 17/305,036

(22) Filed: Jun. 29, 2021

Related U.S. Application Data
(60) Provisional application No. 63/068,841, filed on Aug. 21, 2020.

(30) Foreign Application Priority Data
Jul. 2, 2020 (IN) 202011028207

(51) Int. Cl. G16H 40/63; G16H 40/67; G16H 40/20; G16H 10/60

(52) U.S. Cl. CPC G16H 40/63; G16H 10/60; G16H 40/20; G16H 40/67

(57) ABSTRACT

Various techniques for facilitating server-initiated transmission of messages to medical devices are described herein. For example, a hospital server may transmit strictly regulated clinical requests or messages to medical devices to facilitate clinical operations performed on the medical devices. In addition, the hospital server may transmit requests or messages that are unregulated (or regulated to a lesser degree) to the medical devices to facilitate operations unrelated to the clinical operations of the medical devices. By allowing both regulated and unregulated communications and by allowing both medical-device-initiated and server-initiated uses of the input and output devices provided on the medical devices, faster response times and increased efficiency can be achieved in a hospital environment.

### INCORPORATION BY REFERENCE TO ANY PRIORITY APPLICATIONS

This application claims priority to Indian Patent Application No. 202011028207, filed on Jul. 2, 2020 and titled “SERVER-INITIATED TRANSMISSION OF MESSAGES TO MEDICAL DEVICES,” and U.S. Provisional Application No. 63/068,841, filed on Aug. 21, 2020 and titled “SERVER-INITIATED TRANSMISSION OF MESSAGES TO MEDICAL DEVICES,” the disclosures of which are incorporated herein by reference in their entirety. Any and all applications for which a foreign or domestic priority claim is identified in the Application Data Sheet as filed with the present application are hereby incorporated herein by reference in their entirety under 37 CFR 1.57.

### TECHNICAL FIELD

This disclosure relates to the field of medical devices, and particularly to techniques for transmitting messages to medical devices.

### BACKGROUND

Medical devices capable of performing various clinical operations are commonplace in modern hospital environments. Such medical devices may connect to a hospital network and receive various instructions to perform the clinical operations from a server over the hospital network. Such medical devices may also store rules that govern the clinical operations available on the medical devices to improve patient safety.

### SUMMARY

Various techniques for facilitating server-initiated transmission of messages to medical devices are described herein. For example, a hospital server may transmit strictly regulated clinical requests or messages to medical devices to facilitate clinical operations performed on the medical devices. In addition, the hospital server may transmit requests or messages that are unregulated (or regulated to a lesser degree) to the medical devices to facilitate operations unrelated to the clinical operations of the medical devices. These and other embodiments are described in greater detail below with reference to FIGS. 1-5.

As described herein, allowing both regulated and unregulated communications and allowing both medical-device-initiated and server-initiated uses of the input and output devices provided on the medical devices result in many technical advantages such as faster response times and increased efficiency. Although many of the examples are described in the context of a hospital environment, the techniques described herein can be applied to any environment in which medical devices can operate. The medical devices described herein may be infusion pumps, other medical devices, or any combinations thereof.

### BRIEF DESCRIPTION OF THE DRAWINGS

The embodiments described herein are illustrated by way of example, and not by way of limitation, in the figures of the accompanying drawings in which like references indicate similar elements.

FIG. 1 is a schematic diagram of an example hospital environment including one or more medical devices in accordance with aspects of this disclosure.

FIG. 2 is a block diagram illustrating components of an example hospital environment in accordance with aspects of the present disclosure.

FIG. 3 is a block diagram illustrating a general architecture of an example medical device in accordance with aspects of this disclosure.

FIG. 4 is a process flow diagram illustrating the interactions between a hospital server and a medical device in an example hospital environment in accordance with aspects of this disclosure.

FIG. 5 is a flow chart illustrating an example server-initiated message transmission method in accordance with aspects of this disclosure.

### DETAILED DESCRIPTION

### Introduction

Medical devices are typically equipped with output devices such as display monitors (for outputting visual information or alerts) and speakers (for outputting audio information or alerts), and input devices such as buttons or touch-sensitive display screens for allowing users at the medical devices to input information. However, the use of the input and output devices are primarily initiated by the medical devices themselves to facilitate their own clinical operations. In other words, the determination to utilize the input and output devices is made atomically by the medical devices themselves. For example, upon receiving a user command to initiate an infusion therapy, a medical device may output a confirmation message on the display to confirm that the user wishes to proceed with the infusion therapy. As another example, upon detecting that the battery level is low, the medical device may output beeping sounds to let the user know that the battery level is low and that the user should connect the medical device to a power supply.

However, the use of such input and output devices need not be limited to those situations in which the medical devices need them to facilitate their own clinical operations. For example, there may be situations where it is desirable for the hospital server to cause a message to be displayed on the medical devices in the hospital network or seek input from the user at one of the medical devices. For example, when a doctor decides to discontinue an infusion therapy, the hospital server may notify the nurse in charge of overseeing the infusion therapy at the nurses station and prompt the nurse to head over to the infusion pump to stop the infusion therapy. By the time the nurse sees the message, gets to the infusion pump, and turns off the infusion pump, several hours may have passed. However, if the hospital server were allowed to cause the medical device to display a message to let the user at the infusion pump know that the infusion therapy should be discontinued, the person at the infusion pump may see the message and call the nurse to turn off the infusion pump. Thus, an improved method of allowing the hospital server initiate certain uses of the input and output devices of the medical devices is desired.

Further, hospital servers may send information to the medical devices to facilitate certain clinical operations. For example, a hospital server may send an auto-programming request to an infusion pump within the hospital network to automatically configure the infusion pump for an infusion therapy (e.g., by sending the infusion pump certain information about the infusion therapy such as the type of drug, volume to be infused, duration, etc.). This type of data communication is strictly regulated (e.g., by a governing entity such as the U.S. Food and Drug Administration).

However, by allowing the hospital server to transmit non-regulated messages or messages that are regulated to a lesser degree to the medical devices (e.g., due to the nature of such messages being unrelated to the clinical operations of the medical devices), the input and output devices of the medical devices can be used as a separate messaging channel used to convey and gather information to and from the medical devices. For example, when the hospital server detects that the medical device has moved near a new patient that the medical device is not associated with in the hospital records, the hospital server may request additional information about the new patient from the medical device. The hospital server may transmit a message to the medical device for presentation on the display of the medical device, where the message asks the user at the medical device to confirm that the medical device should be associated with the patient in the hospital records. The user may input an answer into the medical device, and the answer may be transmitted to the hospital server, and the hospital records may be updated according to the answer. Such communication between the hospital server and the medical device does not directly affect any clinical operations being performed on the medical device and thus may not be regulated by a governing entity. However, such communication facilitates the updating of the hospital records, which would otherwise be done manually (e.g., by using a scanner to scan the patient's wrist band and the medical device to associate them in the medical records, etc.). Thus, an improved method of allowing the hospital server to transmit unregulated messages to the medical devices is desired.

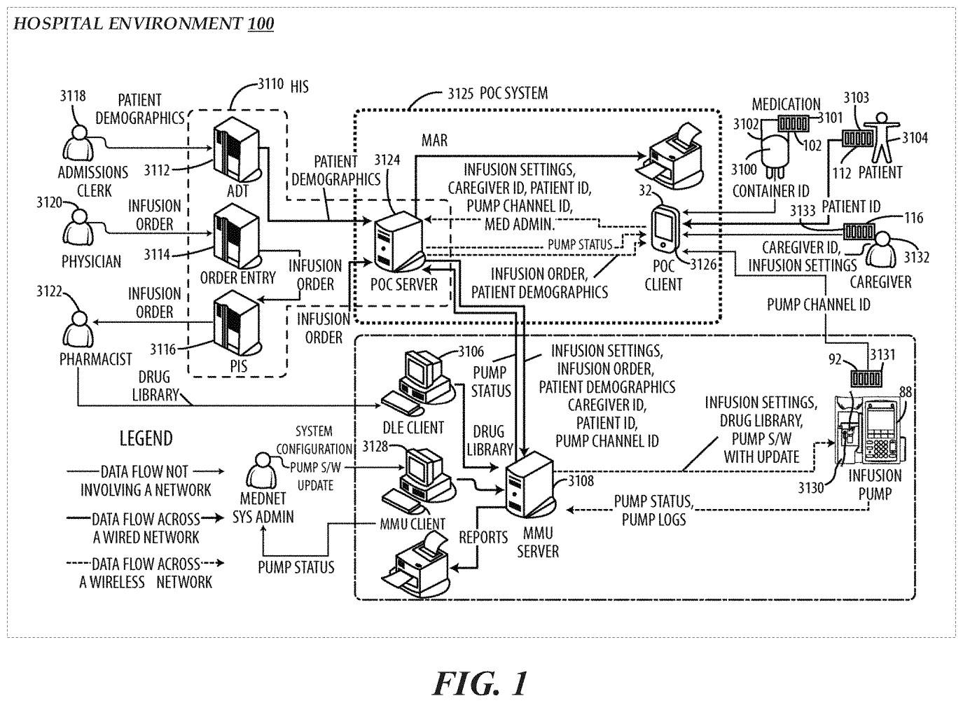

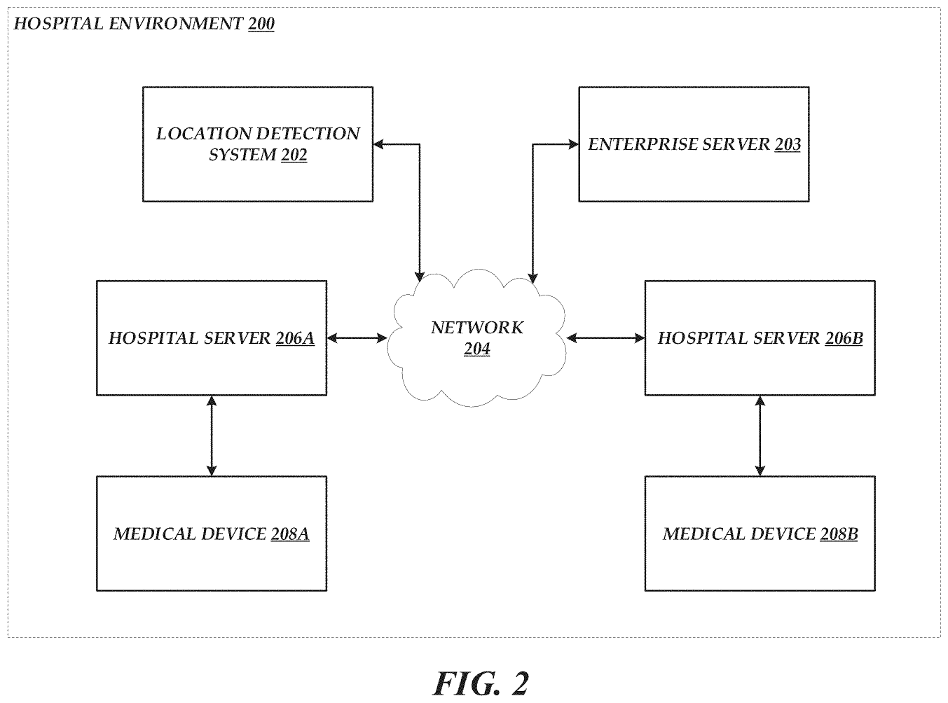

With reference to FIG. 1, an example hospital environment in which one or more of the server-initiated message transmission techniques of the present disclosure may be utilized is described. Following the discussion of FIG. 1, specific details of the various embodiments of the present disclosure are described with reference to FIGS. 2-5.

### Overview of Example Hospital Environment

FIG. 1 illustrates one embodiment of a system for administering medication via an infusion pump in a hospital environment 100. The medication management system (MMS) shown in FIG. 1 includes a medication management unit (MMU) server 3108 and a medical device, such as infusion pump 3130, operating in conjunction with one or more information systems or components of a hospital environment.

Intravenous (IV) fluid(s) and/or medication(s) 3100 in containers 3102 may be administered to a patient 3104 using the system shown in FIG. 1. Although the system shown in FIG. 1 utilizes barcodes and a barcode reader as apparatus to input and read machine-readable information, those skilled in the art will appreciate that other apparatus for reading or inputting information may be utilized. Moreover, a point of care (POC) client 3126 may include an identification receiver 32 adapted to recognize such indicia may be provided in the MMS.

In certain aspects, the IV fluids and/or medications 3100 in container 3102 may be provided with new or supplemental labels with a unique infusion order identifying barcode by a pharmacist according to certain hospital practices. Specifically, drug container specific identification information, such as barcoded information on the container 3102 may include patient identification information, medication identification information, universal identification information, medical device delivery information, and/or medication order information. The IV fluids and/or medications 3100 in barcode-identified containers 3102 may be supplied to hospitals by various vendors, with preexisting unique barcode identifiers, which include medication information and other information, such as a National Disease Center (NDC) code, expiration information, drug interaction information, and the like.

In some aspects of the disclosure, the universal identification information on the container 3102 may be a unique medication order identifier that, by itself, identifies the order associated with the container. In other aspects, the identification information on the container 3102 may be a composite patient/order code that contains both a patient ID (such as a medical record number) and an order ID unique only within the context of the patient. In certain aspects, the identification information on the container 3102 may include a medication ID. The system identified in FIG. 1 may include a drug library editor (DLE) client 3106, such as a notebook, desktop or server computer. The DLE client 3106 may include DLE software. As described above, the MMU server 3108 may have MMU software that is installed and runs on the MMU server 3108. The drug library and other databases may be stored on the MMU server 3108, on a separate server, and/or in remote locations.

Hospital information systems (HIS) 3110 may include one or more computers connected by cabling, interfaces, and/or Ethernet connections. Alternatively, wireless connections and communications may be used in whole or in part. Servers provide processing capability and memory for storage of data and various application programs or modules, including but not limited to an admissions-discharge-and-transfer (ADT) module or computer 3112, a computerized physician order entry (CPOE) module or computer 3114, and a pharmacy information system (PIS) module or computer 3116. Hospital personnel, such as admission clerks 3118, physicians 3120, and pharmacists 3122, respectively, may be authorized to access these modules through client workstations connected to the servers in order to enter data, access information, run reports, and complete other tasks.

In the embodiment shown in FIG. 1, the HIS 3110 may also include a POC system 3125 including a server or POC computer 3124 (sometimes referred to as a barcode point of care server or computer), or the POC computer 3124 may be separate from the HIS 3110. The POC computer 3124 may act as a part of the POC system 3125 (sometimes referred to as the barcode point of care system or BPOC) and may be able to wirelessly communicate through a plurality of wireless communication nodes located throughout the hospital, utilizing a wireless communications protocol, such as IEEE 801.11, IEEE 802.11, or Bluetooth. The POC computer 3124 may communicate wirelessly with a portable thick client, POC client 3126, carried by a caregiver. The POC client 3126 may be a personal digital assistant (PDA) that includes significant memory, display, and processing capabilities. The POC client device may execute a variety of programs stored in its memory in some degree independently of the POC computer 3124.

In one embodiment of FIG. 1, the MMU server 3108 may be hard-wired to the DLE client 3106 and to a MMU client 3128. Alternatively, the MMU and DLE client functions may be combined onto a single client computer/workstation or may reside together with the MMU server 3108 on a single combined MMU/DLE server. The MMU server 3108 may reside in a location remote from the patient's room or treatment area. For instance, the MMU server 3108 may reside in a secure, climate controlled information technology room with other hospital servers, and computer equipment and its client terminals may be located in the pharmacy, biomedical engineering area, nurse station, or ward monitoring area. One MMU server 3108 may monitor, coordinate, and communicate with many infusion pumps 3130. For example, in one embodiment, the MMU software running on the MMU server 3108 may support up to one thousand infusion pumps concurrently.

In embodiment of FIG. 1, the POC client 3126 in the POC system 3125 may communicate through the POC server 3124 with the MMU server 3108. The MMU server 3108 may interface or communicate wirelessly with the infusion pump 3130 through the same wireless nodes utilized by the POC system 3125 and a connectivity engine and antenna on or in the infusion pump 3130. Communication between the infusion pump 3130 and the POC client 3126 may take place through the MMU server 3108 and POC server 3124. The MMU server 3108 may store in an associated memory both the logical ID and the network ID or Internet Protocol (IP) address of the infusion pump(s) 3130, such that only the MMU server 3108 may communicate in a direct wireless manner with the infusion pump 3130. Alternatively, the MMU server 3108 may provide the IP address and other information about the infusion pump 3130 to the POC system 3125 to facilitate direct communication between the POC system 3125 and the infusion pump 3130.

Upon admission to the hospital, the admission clerk 3118 or similar personnel may enter demographic information about each patient 3104 into an associated memory of the ADT module or computer 3112 of an HIS database stored in an associated memory of the HIS 3110. Each patient 3104 may be issued a patient identification wristband, bracelet, or tag 112 that may include an identifier 3103, such as a barcode or RFID tag, identifying the patient. The wristband, bracelet, or tag 112 may also include other information, in machine readable or human-readable form, such as the name of the patient's doctor, blood type, allergies, and the like.

The patient's doctor 3120 may prescribe medical treatment by entering a medication order into the CPOE module or computer 3114 within the HIS 3110. The medication order may specify a start time, stop time, a range of allowable doses, physiological targets, route, and site of administration. In the case of an order for infusion of fluids or medication, the order may be written in various formats, and may include the patient's name, patient ID number, a unique medication order or prescription number, a medication name, medication concentration, a dose or dosage, frequency, and/or a time of desired delivery. This information may be entered into the memory of the CPOE module or computer 3114, and may be stored in a memory associated with at least the POC server 3124.

The medication order may also be delivered electronically to the PIS module or computer 3116 in the pharmacy and may be stored in an associated memory. The pharmacist 3122 may screen the prescribed order, translate it into an order for dispensing medication, and prepare the medication or fluids with the proper additives and/or necessary diluents. The pharmacist 3122 may prepare and affix a label 102 with drug container specific identifying information 3101 to the medication or drug container 3102. The label may include in machine-readable and/or human-readable form medical device specific delivery information including but not limited to the dispense ID number, patient ID, drug name, drug concentration, container volume, volume-to-be-infused (“VTBI”), rate, duration, and the like. Only two of the three variables VTBI, rate, and duration may be defined as the third may be calculated when the other two are known. The labeled medication may be delivered to a secure, designated staging location or mobile drug cart on the ward or floor near the patient's room or treatment area. The medication order pending dispensing or administration may be posted to a task list in the HIS 3110 and POC system 3125 and stored in an associated memory.

The caregiver 3132 (e.g., a nurse) may use the identification receiver 32 associated with the POC client 3126 to scan his/her caregiver identification badge 116 and enter a password, which logs the caregiver into the system and authorizes the caregiver to access a nurse's task list from the POC system 3125 through the POC client 3126. The caregiver 3132 may view from the task list that IV drugs are to be administered to certain patients 3104 in certain rooms. The caregiver 3132 obtains the necessary supplies, including medications, from the pharmacy and/or a staging area in the vicinity of the patient's room.

The caregiver 3132 may take the supplies to a patient's bedside, turn on the infusion pump 3130, verify that the network connection icon on the infusion pump 3130 indicates a network connection (for example, a wireless connection such as Wi-Fi or the like) is present, select the appropriate clinical care area (CCA) on the infusion pump 3130, and mount the IV bag, container, or vial 3102 and any associated tube set as required in position relative to the patient 3104 and infusion pump 3130 for infusion. Another connection icon on the infusion pump 3130 or pump user interface screen can indicate that a wired or wireless connection to the MMU server 3108 is present. Using the identification receiver/reader integral to the POC client 3126, the caregiver 3132 may scan the barcode on the patient's identification wristband, bracelet, or tag 112 or other patient identification device. A task list associated with that particular patient may appear on the POC client 3126 screen. The task list, which may also include orders to give other forms of treatment or medication by other routes (oral, topical, etc.), may be obtained from the HIS 3110 via the POC server 3124 and communicated wirelessly to the POC client 3126. In one embodiment, the list is generated by matching the scanned patient ID with the patient ID for orders in memory within the POC server 3124. In another embodiment, the order information may be obtained by scanning the drug container specific identification information for associated orders in memory within the POC server 3124, through the following step(s).

The caregiver 3132 may scan the medication barcode label 102 containing medication container specific identification information 3101 on the medication container 3102 with the POC client 3126. The POC client 3126 may highlight the IV administration task on the task list and send the scanned medication container specific identification information, such as dispense ID information, from the medication container 3102, to the POC server 3124. The POC server may use the medication container specific identification information to pull together the rest of the order details and send them back to the POC client 3126. The POC client 3126 may then display an IV Documentation Form on its screen. One side of the IV Documentation Form screen may show the order details as “ordered” and the other side may be reserved for a status report from the infusion pump 3130. The status report from the infusion pump 3130 may be transmitted to the POC client 3126 through the POC server 3124 and MMU server 3108. The lower portion of the IV Documentation Form screen may provide the caregiver 3132 with instructions (like to scan the infusion pump 3130 barcode) or identify whether the pump is running or stopped.

The caregiver 3132 may then scan the barcode label 92 associated with the infusion pump 3130 (or pump channel if the pump is a multi-channel pump). The barcode label 92 may contain medical device specific identification information 3131, such as the logical name and/or logical address of the device or channel. The POC system 3125 then automatically bundles the information into a program pump request containing the “order details” and in one embodiment, without further interaction with the caregiver 3132, transmits this information to the MMU server 3108.

The program pump request may include at least some of the following information (in HIS/POC system format): a Transaction ID, which may include a Logical Pump ID, a Pump Compartment, a Pump Channel ID, a Reference Device Address, a Caregiver ID, a Caregiver Name, a Patient/Person ID (HIS identifier), a Patient Name, a Patient Birth Date & Time, a Patient Gender, a Patient Weight, a Patient Height, and an Encounter ID which may include a Room, a Bed, and a Building (including CCA). The program pump request may also include Order Information or “order details”, including an Order ID, a Start Date/Time, a Stop Date/Time, a Route of Administration, a Rate, a Duration of Infusion (Infuse Over), a Total Volume to be Infused (VTBI), an Ad Hoc Order Indicator, and Ingredients including HIS Drug Name or HIS Generic Drug Name, HIS Drug Identifier or HIS Generic Drug ID, Rx Type (Additive or Base), Strength w/units, and Volume w/units. The program pump request may further include Patient Controlled Analgesia (PCA) Orders Only information, such a PCA Mode-PCA only, Continuous only, or PCA and Continuous, a Lockout Interval (in minutes), a PCA Continuous Rate, a PCA Dose, a Loading Dose, a Dose Limit, a Dose Limit Time w/ units, a Total Volume in vial or syringe, and Order Comments.

The MMU server 3108 may map or convert the wide range of expressions of units allowed by the HIS 3110 or POC system 3125 for POC client 3126 requests into the much more limited set of units allowed in the MMU server 3108 and infusion pump 3130. For example, the POC client 3126 request may express “g, gm, gram, or grams” whereas the MMU server 3108 and/or infusion pump 3130 may accept “grams” only. Infusion pump 3130 delivery parameters or infusion pump 3130 settings are mapped or converted from corresponding order information or “order details” of the program pump request.

The MMU server 3108 may store in an associated memory a mapping or translation table that keep track of the logical ID, serial number or other identifier of an infusion pump 3130 and the corresponding current network (static or dynamic) address (Internet Protocol (IP) address) or ID of the infusion pump 3130 on the network, which in this example is a wireless network. The MMU server 3108 may be able to translate or associate a given identifier of the infusion pump 3130 with its network address in the translation table and provide the network IP address to the requesting POC system 3125 or device. The MMU server 3108 may also store in an associated memory and/or look up the drug library applicable to the scanned infusion pump 3130 and/or convert the Drug ID and Strength from the pump program request into an index number of the medication at the desired strength or concentration from the drug library. The duration of the infusion may come from the POC system 3125 in hours and minutes and may be converted to just minutes for the infusion pump 3130 to recognize it. Volume or VTBI may be rounded to provide a value-specific and infuser-specific number of digits to the right of the decimal point. Units (of drug) may be converted to million units where appropriate. Patient weight may be converted and either rounded according to infuser-specific rules or not sent to the infuser.

Once the MMU server 3108 transforms the information from the program pump request into infusion pump settings or delivery parameters and other information in a format acceptable to the infusion pump 3130, the MMU server 3108 may wirelessly download a command message to the infusion pump 3130. If the infusion pump 3130 is not already equipped with the latest appropriate version of the hospital-established drug library, the MMU server 3108 may also automatically download a drug library to the infusion pump 3130. The hospital-established drug library may be maintained in a separate process undertaken by the biomedical engineer or pharmacist 3122 to place limits on the programming of the infusion pump 3130, as well as other infusion pump operating parameters such as default alarm settings for air in the line, occlusion pressure, and the like. The drug library may set up acceptable ranges or hard and/or soft limits for various drug delivery parameters in the infusion pump 3130.

The MMU server 3108 may also download to the infusion pump new versions, patches, or software updates of the infusion pump's internal operating system software. The infusion settings or delivery parameters and other information from the MMU server 3108 may be entered into the memory of the infusion pump 3130 and the infusion pump 3130 settings may automatically populate the programming screen(s) of the infusion pump 3130, just as if the caregiver 3132 had entered the information and settings manually. The infusion pump 3130 screen may populate with the name of the drug and drug concentration based on the drug library index number, patient weight, rate, VTBI, and/or duration. Further, the MMU server 3108 may detect that certain conditions are satisfied and transmit messages to the infusion pump to cause the infusion pump to output such messages via the output devices of the infusion pump and collect and relay any input from the user at the infusion pump via the input devices of the infusion pump, as described in greater detail below with reference to FIGS. 2-5. A return message of confirmation signal may be sent to the MMU server 3108 by the infusion pump 3130 to indicate that the command message has been received. At this point, if necessary, the caregiver 3104 may manually enter any additional infusion settings or optional information that was not included in the command message.

The infusion pump 3130 may then prompt the caregiver 3132 to start the infusion pump 3130 by pressing the start button. When the caregiver 3132 presses the start button, a confirmation screen with the infusion settings programmed may be presented for confirmation and an auto-program acknowledgment message can be sent to the MMU server 3108 to forward without request (i.e., pushed in a near real-time manner) or provide to the POC system 3125 when requested or polled. When the caregiver 3132 presses the button to confirm, the infusion pump 3130 may begin delivering fluid according to the programmed settings. The infusion pump 3130 may send a status message to the MMU server 3108 indicating that the infusion pump 3130 was successfully auto-programmed, confirmed and started by the caregiver 3132, and is now delivering fluid. This information may also be displayed at the infusion pump. The MMU server 3108 may continue to receive logs and status messages wirelessly from the infusion pump 3130 periodically as the infusion progresses or when alarms occur.

The MMU server 3108 may report a portion of the initial status message to the POC client 3126 through the POC server 3124 (in MMU format) to indicate that the infusion pump 3130 has been auto-programmed and the caregiver 3132 has confirmed the settings. The MMU server 3108 may communicate to the POC system 3125 and/or at the infusion pump 3130 the actual Rate, VTBI, and Duration. A notation at the bottom of the screen of the POC client and/or the infusion pump may indicate that the infusion pump 3130 is running. The infusion pump 3130 may compare and give a visual, audio, or other type of affirmative signal if the pump information matches or acceptably corresponds with the ordered information. An initial determination of whether the pump information matches the order may be done in the MMU server 3108 and communicated to the POC client 3126 through the POC server 3124. Alternatively, the POC server 3124 or the infusion pump 3130 may make the necessary comparisons. If the pump information does not match the order, the infusion pump 3130 at the display 88 may output a visual, audio, or other type of negative signal, which may include an error message.

The caregiver 3132 may be prompted to review and press a save button on the infusion pump 3130 if the order has been begun as desired or any variations are acceptable. The MMU server 3108 may receive status, event, differences, and variation information from the infusion pump 3130 and pass such information to the POC system 3125. In a separate subsequent step, the nurse may electronically sign the record and presses a send button on the POC client POC client 3126 to send the information to the patient's electronic medication record (EMR) or medication administration record (MAR).

### Other Environments

FIG. 1 illustrates one example environment in which the various server-initiated message transmission techniques of the present disclosure may be utilized. However, the embodiments described herein are not limited to such an environment, and may be applied to any network environment including one or more servers in which medical devices perform clinical operations. An example system that may be implemented in one or more of such network environments to provide server-initiated message transmission is described below with reference to FIG. 2.

### System Overview

FIG. 2 is a block diagram of an example hospital environment 200, which includes an arrangement of computer hardware and software components that may be used to implement aspects of the present disclosure. The hospital environment 200 may include many more (or fewer) elements and/or sub-elements than those shown in FIG. 2. It is not necessary, however, that all of these elements be shown in order to provide an enabling disclosure. As illustrated in FIG. 2, the hospital environment 200 includes a location detection system 202, an enterprise server 203, a hospital server 206A, and a hospital server 206B connected to a network 204, and additionally, a medical device 208A in communication with the hospital server 206A, and a medical device 208B in communication with the hospital server 206B. Although only two hospital servers and two medical devices are shown in FIG. 2, the hospital environment 200 may, in some embodiments, include only a single hospital server or more than two hospital servers. Additionally or alternatively, one or more of the hospital servers in the hospital environment 200 may include two or more medical devices.

### Location Detection System

The location detection system 202 may use Global Navigation Satellite System (GNSS), such as Global Positioning System (GPS) or GLONASS navigation system, for geo-spatial positioning, and/or non-GNSS technologies such as Pedestrian Dead Reckoning (PDR), inertial navigational systems, magnetic positioning systems, and the like. In some embodiments, the location detection system 202 uses existing wireless technologies to perform geo-positioning, which may include Wi-Fi-based positioning systems (WPS), Bluetooth-based positioning systems, Radio-Frequency Identification (RFID) systems, and others. In other embodiments, the location detection system may include video processing systems, ultrasound-based systems, visible light communication systems, and so forth.

The location detection system 202 can allow geo-fences to be drawn on a map and notify the enterprise server 203 when a medical device crosses the geo-fences (e.g., when the medical device enters a geo-fenced area, exits a geo-fenced area, or both). If a hospital administrator wishes to take certain actions (e.g., track the locations of medical devices, transmit messages to the medical devices, request additional information from the user(s) at the medical device(s), etc.) in response to medical devices entering or exiting specific areas of the hospital (e.g., rooms, floors, wings, buildings, or clinical care areas such as emergency room, operating room, intensive care unit, etc.), he or she may configure the location detection system 202 to monitor movements of medical devices across the boundaries of such areas. In response to the notification from the location detection system 202 that a medical device has entered a designated geo-fenced area, the enterprise server 203 may transmit further instructions, for example, to the hospital server in communication with the medical device.

In some embodiments, the location detection system 202 also detects how the medical devices entered or exited a geo-fenced area, and different actions may be triggered depending on the manner in which the medical device has entered or exited the geo-fenced area. For example, the enterprise server or the hospital server may take one action if an infusion pump exited the hospital through the front door and another action if the infusion pump exited on an ambulance. In other embodiments, the location detection system 202 is omitted from the hospital environment 200. In such embodiments, the individual medical devices may be configured to determine and report their locations to the hospital servers, and the hospital servers may take location-based actions described herein.

### Enterprise Server

The enterprise server 203 may be a server in charge of the entire hospital or enterprise that can communicate with all hospital servers in the hospital or enterprise (e.g., the hospital environment 200). The enterprise server 203 may send instructions to the location detection system 202 to identify the medical devices that the location detection system 202 should monitor and to define the gen-fencing boundaries that the enterprise server 203 should be notified about. In response to a notification from the location detection system 202 indicating that a monitored medical device has entered a new geo-fenced area, the enterprise server 203 may take certain designated actions such as log the location change of the medical device, instruct the hospital server connected to the medical device to transmit certain messages to the medical device, and the like. In some embodiments, the enterprise server 203 is omitted, and the location detection system 202 communicates directly with one or more of the hospital servers in the hospital environment 200.

In some embodiments, the enterprise server 203 (or the hospital server if location change notification is sent directly to the hospital server) may determine whether the location detection system 202 is an authorized, authenticated service prior to initiating any location-based actions described herein. The enterprise server 203 may utilize OAuth or another authorization protocol such as a public/private key certificate exchange.

### Network

The network 204 may be any wired network, wireless network, or combination thereof. In addition, the network 204 may be a personal area network, local area network, wide area network, over-the-air broadcast network (e.g., for radio or television), cable network, satellite network, cellular telephone network, or combination thereof. For example, the network 204 may be a publicly accessible network of linked networks such as the Internet. For example, the communications between the location detection system 202 and the enterprise server 203 may be over a publicly accessible network of linked networks such as the Internet, and the communications between the enterprise server 203 and the hospital servers 206A and 206B (and also the communications between the hospital server 206A and the medical device 208A, and the communications between the hospital server 206B and the medical device 208B) may be implemented on one or more wired and/or wireless private networks. The enterprise server 203 may be a cloud server that includes a collection of services, which are delivered via the network 204 as web services.

### Hospital Server

The hospital servers 206A and 206B may each represent a version of the MMU server 3108 described with reference to FIG. 1. For example, the hospital server 206A may communicate with the medical devices in Hospital A (e.g., send commands to medical devices to initiate or stop clinical operations, transmit messages to be output by the medical devices, and the like), and the hospital server 206B may communicate with the medical devices in Hospital B that is separate from Hospital A (but may belong to the same hospital network or enterprise as Hospital A).

### Medical Device

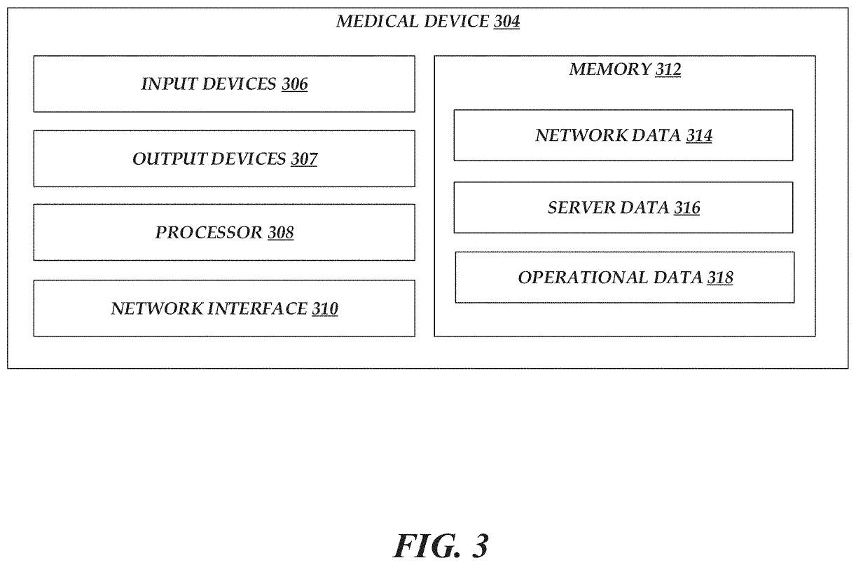

The medical devices 208A and 208B may be any medical device that are mobile and can be moved across the geo-fences monitored by the location detection system 202. For example, the medical devices 208A and 208B can be infusion pumps, patient monitors, and the like. The medical devices 208A and 208B are described in greater detail below with reference to FIG. 3. In other embodiments, the medical devices 208A and 208B can be medical devices permanently mounted or temporarily mounted to a specific location in the hospital.

With reference to FIG. 3, the components of an example medical device are described in greater detail. The example architecture of the medical devices 208A and 208B depicted in FIG. 2 includes an arrangement of computer hardware and software modules that may be used to implement aspects of the present disclosure. The medical device 304 may include many more (or fewer) elements and/or sub-elements than those shown in FIG. 3. It is not necessary, however, that all of these elements be shown in order to provide an enabling disclosure.

As illustrated, the medical device 304 includes input devices 306, output devices 307, a processor 308, a network interface 310, and a memory 312, all of which may communicate with one another by way of a communication bus. The input devices 306 may include devices via which information is received from a user of the medical device 304. For example, the input devices may include a physical or digital keyboard, a touchscreen, a microphone, and the like. The output devices 307 may include devices via which information is presented to the user of the medical device 304. For example, the output devices 307 may include a speaker, a display, an LED, a printer, and the like. For example, the output devices 307 may include a display that can display information generated or stored by the medical device 304 or any other information associated with the medical device 304, or information received from a hospital server over the hospital network. For example, the medical device 304 may be an infusion pump being used to deliver medication to a patient. In such a case, the display may display the volume of the medication infused so far, the volume of the medication to be infused, the rate at which the medication is being infused, and the like. The processor 308 may receive information and instructions from other computing systems or services via a network. The processor 308 may also transmit information to and receive information from the memory 312 and further provide content to the output devices 307 for presentation to the user (e.g., visual content, alarm sound, etc.). The network interface 310 may provide connectivity to one or more networks or computing systems in the network environment described herein. For example, the network interface 310 may be a serial port, a parallel port, or any other communication interface that can enable or facilitate wired or wireless communication according to any communication protocols such as Zigbee (e.g., IEEE 802.15.4), Bluetooth, Wi-Fi (e.g., IEEE 802.11), Near Field Communication (NFC), and the like.

The memory 312 may contain computer program instructions (grouped as modules in some embodiments) that the processor 308 can execute in order to implement one or more aspects of the present disclosure. The memory 312 may include RAM, ROM, and/or other persistent, auxiliary, or non-transitory computer-readable media. In some embodiments, the memory 312 stores an operating system that provides computer program instructions for use by the processor 308 in the general administration and operation of the medical device 304. As illustrated in FIG. 3, the memory 312 may include network data 314, server data 316, and operational data 318. In some embodiments, the medical device 304 uses the network data 314 to connect to a network in the hospital environment (e.g., Wi-Fi network), uses the server data 316 to connect to a hospital server in the hospital environment (e.g., MMU server 3108 of FIG. 1), and uses the operational data 318 to perform one or more clinical operations (e.g., initiate an infusion therapy on a patient). In some embodiments, the operational data may also referred to herein as clinical data or clinical settings.

Although not shown in FIG. 3, the memory 312 may store programs, instructions, modules, libraries, settings, parameters, and/or other types of data that may be used by the medical device 304 to perform its operations. For example, the memory 312 may store location data indicating the current location of the medical device 304. Such location data may be updated in response to the change in the location of the medical device 304, and transmitted to the hospital server and/or the enterprise server for monitoring and logging purposes (e.g., such that various location-based metrics such as device utilization can be generated based on how long the individual medical devices spend in which geographical areas such as hospital rooms, cleaning stations, specific clinical care areas, specific buildings and facilities, etc.).

As another example, the memory 312 may store program code or data used for outputting non-clinical user interfaces in response to instructions from the hospital server (e.g., to display a message such as “return this infusion pump to the biomed” or to prompt request for additional information such as “should this infusion pump be associated with patients A or B?” along with user interface elements “No”, “Patient A”, “Patient B” for user selection).

The medical device 304 may also include any other number of components such as multiple displays, multiple processors, multiple network interfaces, and/or multiple memories. Further, the medical device 304 may include one or more additional storage devices for storing data generated by the medical device 304 or other data utilized in implementing aspects of the present disclosure.

### Server-Initiated Message Transmission Processes

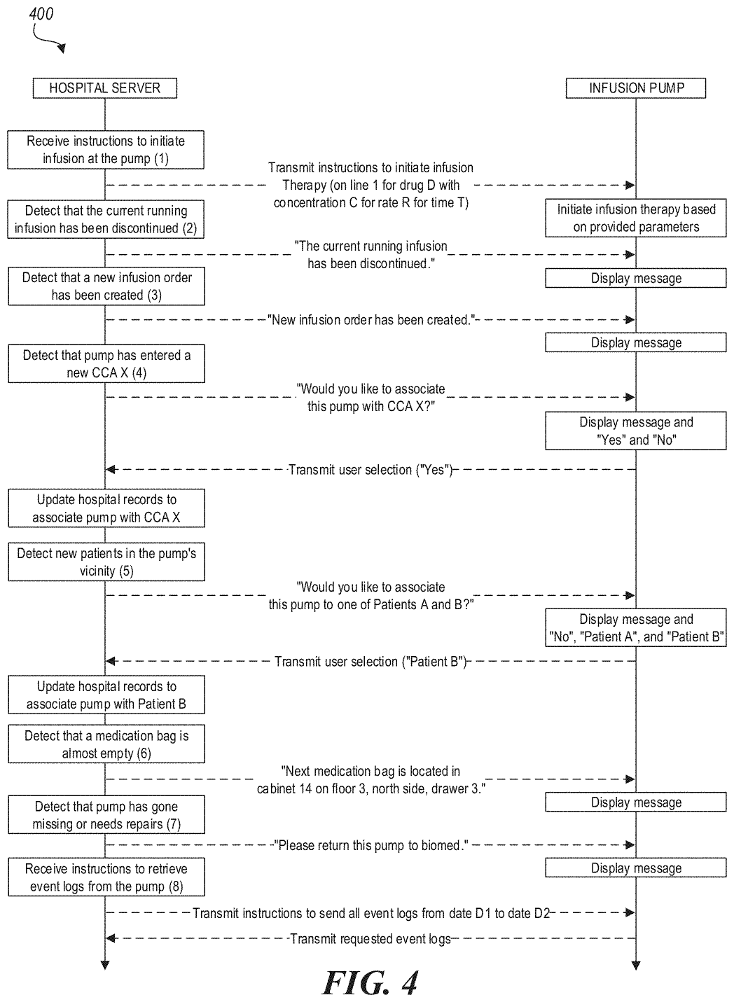

With reference now to FIG. 4, an example server-initiated message transmission processes 400 will be described. As shown, FIG. 4 illustrates examples (1)-(8) of server-initiated message transmission that occur between a hospital server and an infusion pump.

In example (1), the hospital server receives instructions to initiate an infusion therapy at the infusion pump. For example, a doctor may have ordered the infusion therapy for a patient at the infusion pump, and the hospital server may have detected the order in the hospital records system. In response, the hospital server transmits an instruction to initiate or perform the infusion therapy with one or more parameters according to which the infusion therapy is to be performed by the infusion pump (e.g., infusion of drug D on line 1 with concentration C for rate R for time T). In response, the infusion pump initiates the requested infusion therapy based on the provided parameters. In example (1), the hospital server transmits a command to perform a clinical operation (e.g., infusion therapy), and the communication between the hospital server and the infusion pump that causes the infusion pump to perform the clinical operation may be strictly regulated by a governing entity, such as the U.S. Food and Drug Administration. In response to receiving such a regulated clinical request from the hospital server, the infusion pump performs an operation directly related to its clinical operations (e.g., initiate an infusion therapy). Also, in this case, the request from the hospital server directly impacts the infusion pump (e.g., operation status of the infusion pump has changed).

In example (2), the hospital server detects that the currently running infusion therapy has been ordered to be discontinued. For example, a doctor who issued the infusion therapy may have decided to discontinue the infusion therapy and entered an order to discontinue the infusion therapy into the hospital records system, and the hospital server may have detected the order. In response, the hospital server transmits an instruction to output a message (also referred to herein as an indication) to the infusion pump (e.g., “The currently running infusion therapy has been ordered to be discontinued.”). In response, the infusion pump displays the message on the display so that the user at the infusion pump can see the message and discontinue the infusion therapy (e.g., by turning off the infusion pump). In example (2), the infusion pump operates as a “thin” point-of-care device that relays information between the hospital server and the user at the infusion pump, without affecting the clinical operations of the infusion pump or accessing any data specific to the infusion pump. Due to the nature of this communication between the hospital server and the infusion pump, the communication may not be regulated or may be regulated to a lesser degree than the communication in example (1). By allowing both regulated and unregulated communications between the hospital server and the infusion pump, faster response times and increased efficiency can be achieved in the hospital environment. For example, if the nurse responsible for turning off the infusion pump is at the infusion pump, he or she can turn off the infusion pump in response to seeing the message displayed on the screen (or hearing the voice message or beeping sound played via the speaker). If the nurse is at another location, another person at the infusion pump may see or hear the message and call the nurse to turn off the infusion pump. In some embodiments, when a user at the infusion pump takes a certain action in response to the message displayed on the screen of the infusion pump, the infusion pump returns a status message back to the sender of the message (e.g., the hospital server), indicating disposition of the message and/or the action taken by the user (e.g., “proposed option accepted”, “proposed option rejected”, “message ignored”, “pump turned off”, “pump turned on”, “infusion paused”, “infusion canceled”, etc.). By doing so, the infusion pump permits the hospital server (or the sender of the message) to coordinate the message status across many devices, including alarm systems, to prevent redundant actions being taken (e.g., by the user and/or other people at the hospital).

In example (3), the hospital server detects that a new infusion order has been created for the infusion pump. For example, a doctor may have ordered the infusion therapy for a patient at the infusion pump, and the hospital server may have detected the order in the hospital records system. In response, the hospital server transmits an instruction to output a message to the infusion pump (e.g., “A new infusion therapy order has been created.”). In response, the infusion pump displays the message on the display so that the user at the infusion pump can see the message and initiate the infusion therapy (e.g., by inputting various infusion parameters into the infusion pump). In example (3), the infusion pump operates as a “thin” point-of-care device that relays information between the hospital server and the user at the infusion pump, without affecting the clinical operations of the infusion pump or accessing any data specific to the infusion pump. Due to the nature of this communication between the hospital server and the infusion pump, the communication may not be regulated or may be regulated to a lesser degree than the communication in example (1). By allowing both regulated and unregulated communications between the hospital server and the infusion pump, faster response times and increased efficiency can be achieved in the hospital environment. For example, if the nurse responsible for initiating the infusion therapy is at the infusion pump, he or she can initiate the infusion therapy in response to seeing the message displayed on the screen (or hearing the voice message or beeping sound played via the speaker). If the nurse is at another location, another person at the infusion pump may see or hear the message and call the nurse to initiate the infusion therapy.

In example (4), the hospital server detects that the infusion pump has entered a new clinical care area (“CCA A”). For example, a location detection system 202 may have detected that the infusion pump has entered a new geo-fence associated with a clinical care area not associated with the infusion pump, and transmitted this information to the hospital server. In response, the hospital server transmits an instruction to output a message to the infusion pump (e.g., “Would you like to associate this infusion pump with CCA A?”). In response, the infusion pump displays the message along with user interface elements labeled “Yes” and “No.” The “Yes” user interface element may, when selected by the user at the infusion pump, indicate to the hospital server that CCA A is to be associated with the infusion pump, and the “No” user interface element may, when selected by the user at the infusion pump, indicate to the hospital server that CCA A is not to be associated with the infusion pump. In response to the user selection of one of the user interface elements, a corresponding indication is transmitted to the hospital server. In response, the hospital server can take corresponding actions such as causing the hospital records system (e.g., a database maintaining the records relevant to the hospital environment) to be updated based on the user selection made at the infusion pump and/or communicating the indication from the infusion pump to one or more other servers. In some embodiments, instead of the hospital server, a real-time location system (RTLS) detects that the infusion pump has entered a new clinical care area. In such embodiments, in response to an indication received from the infusion pump that the infusion pump is to be associated with the new CCA, the RTLS may forward the indication to the hospital server so that the hospital server can make appropriate changes in the hospital records system and/or further communicate with one or more other servers to take additional action. In example (4), the infusion pump operates as a “thin” point-of-care device that relays information between the hospital server and the user at the infusion pump, without affecting the clinical operations of the infusion pump or accessing any data specific to the infusion pump. For example, even though the hospital server may subsequently cause the infusion pump to update certain settings due to the change in the clinical care area, the communication illustrated in example (4) does not immediately impact the operations of the infusion pump. Due to the nature of this communication between the hospital server and the infusion pump, the communication may not be regulated or may be regulated to a lesser degree than the communication in example (1). By allowing both regulated and unregulated communications between the hospital server and the infusion pump, faster response times and increased efficiency can be achieved in the hospital environment. For example, if a nurse were responsible for manually updating the clinical care area associated with the infusion pump in the hospital records system, he or she may need to undergo cumbersome procedures to update the association in the hospital records system. In example (4), the location change is automatically detected, and the nurse at the infusion pump simply needs to select “Yes” on the infusion pump to update the association.

In example (5), the hospital server detects new patients that are not associated with the infusion pump in the infusion pump's vicinity. For example, a location detection system 202 may have detected patients not currently associated with the infusion pump in the vicinity of the infusion pump, and transmitted this information to the hospital server. In response, the hospital server transmits an instruction to output a message to the infusion pump (e.g., “Would you like to associate this infusion pump with Patients A or B?”). In response, the infusion pump displays the message along with user interface elements labeled “No,” “Patient A,” and “Patient B.” The “No” user interface element may, when selected by the user at the infusion pump, indicate to the hospital server that neither of the patients should be associated with the infusion pump, the “Patient A” user interface element may, when selected by the user at the infusion pump, indicate to the hospital server that Patient A should be associated with the infusion pump, and the “Patient B” user interface element may, when selected by the user at the infusion pump, indicate to the hospital server that Patient B should be associated with the infusion pump. In response to the user selection of one of the user interface elements, a corresponding indication is transmitted to the hospital server. In response, the hospital server can take corresponding actions such as causing the hospital records system (e.g., a database maintaining the records relevant to the hospital environment) to be updated based on the user selection made at the infusion pump and/or communicating the indication from the infusion pump to one or more other servers. In some embodiments, instead of the hospital server, an RTLS detects a new patient not currently associated with the infusion pump in the vicinity of the infusion pump. In such embodiments, in response to an indication received from the infusion pump that the infusion pump is to be associated with the new patient, the RTLS may forward the indication to the hospital server so that the hospital server can make appropriate changes in the hospital records system and/or further communicate with one or more other servers to take additional action. In example (5), the infusion pump operates as a “thin” point-of-care device that relays information between the hospital server and the user at the infusion pump, without affecting the clinical operations of the infusion pump or accessing any data specific to the infusion pump. Due to the nature of this communication between the hospital server and the infusion pump, the communication may not be regulated or may be regulated to a lesser degree than the communication in example (1). By allowing both regulated and unregulated communications between the hospital server and the infusion pump, faster response times and increased efficiency can be achieved in the hospital environment. For example, if a nurse were responsible for manually updating the patient associations for the infusion pump in the hospital records system, he or she may need to undergo cumbersome procedures to update the patient association in the hospital records system. In example (5), the infusion pump's proximity to one or more patients is automatically detected, and the nurse at the infusion pump simply needs to select “Patient A” or “Patient B” on the infusion pump to update the patient association.

In example (6), the hospital server detects that a medication bag of a multi-bag infusion therapy is almost empty (e.g., fallen below a threshold level of medication volume). For example, a sensor configure to monitor the volume levels in medication bags may have detected that the volume level in the medication bag has reached a threshold level of medication volume during a multi-bag infusion therapy, and transmitted this information to the hospital server. In response, the hospital server transmits an instruction to output a message to the infusion pump (e.g., “The next medication bag for this multi-bag infusion therapy is located in cabinet 14 on floor 3, north side, drawer 3.”). In response, the infusion pump displays the message on the display so that the user at the infusion pump can see the message and retrieve the next medication bag for the multi-bag infusion therapy (e.g., by going to the described location and getting the medication bag). In example (6), the infusion pump operates as a “thin” point-of-care device that relays information between the hospital server and the user at the infusion pump, without affecting the clinical operations of the infusion pump or accessing any data specific to the infusion pump. Due to the nature of this communication between the hospital server and the infusion pump, the communication may not be regulated or may be regulated to a lesser degree than the communication in example (1). By allowing both regulated and unregulated communications between the hospital server and the infusion pump, faster response times and increased efficiency can be achieved in the hospital environment. For example, if the nurse responsible for changing the medication bag is at the infusion pump, he or she can retrieve the next medication bag for the multi-bag infusion therapy in response to seeing the message displayed on the screen (or hearing the voice message or beeping sound played via the speaker). If the nurse is at another location, another person at the infusion pump may see or hear the message and call the nurse to retrieve the next medication bag.

In example (7), the hospital server detects that the infusion pump has gone missing or needs repairs. For example, the hospital server may have detected that the infusion pump has been malfunctioning or outputting errors (e.g., by monitoring the behavior of the infusion pump over the hospital network or in response to an indication received from the infusion pump). In response, the hospital server transmits an instruction to output a message to the infusion pump (e.g., “Please return this infusion pump to biomed.”). In response, the infusion pump displays the message on the display so that the user at the infusion pump can see the message and return the infusion pump to a biomedical equipment technician on site. In example (7), the infusion pump operates as a “thin” point-of-care device that relays information between the hospital server and the user at the infusion pump, without affecting the clinical operations of the infusion pump or accessing any data specific to the infusion pump. Due to the nature of this communication between the hospital server and the infusion pump, the communication may not be regulated or may be regulated to a lesser degree than the communication in example (1). By allowing both regulated and unregulated communications between the hospital server and the infusion pump, faster response times and increased efficiency can be achieved in the hospital environment. For example, if a biomedical equipment technician were responsible for personally tracking down and retrieving the infusion pump, he or she may not know the exact location of the infusion pump, and the infusion pump may not be examined in a timely manner. In example (7), the user at the infusion pump can see the message and allow the infusion pump to be returned to the biomedical equipment technician in a timely manner.

In example (8), the hospital server receives instructions to retrieve event logs from the infusion pump. For example, an administrator may have entered a request for the event logs stored on the infusion pump, and the hospital server may have detected the request in the hospital records system. In response, the hospital server transmits an instruction to send the event logs stored on the infusion pump (e.g., all event logs from date D1 to date D2). In response, the infusion pump transmits the requested event logs to the hospital server. In example (8), the hospital server transmits a command to transmit the event logs stored on the infusion pump to the hospital server. Retrieval of data generated by medical devices may be strictly regulated by a governing entity, such as the U.S. Food and Drug Administration. In response to receiving such a regulated request from the hospital server, the infusion pump performs an operation directly related to its clinical operations (e.g., logs indicating the details of the infusion therapies performed by the infusion pump). Also, in this case, the request from the hospital server directly impacts the infusion pump (e.g., data stored on the infusion pump is retrieved and transmitted to the hospital server).

### Example Server-Initiated Message Transmission Method

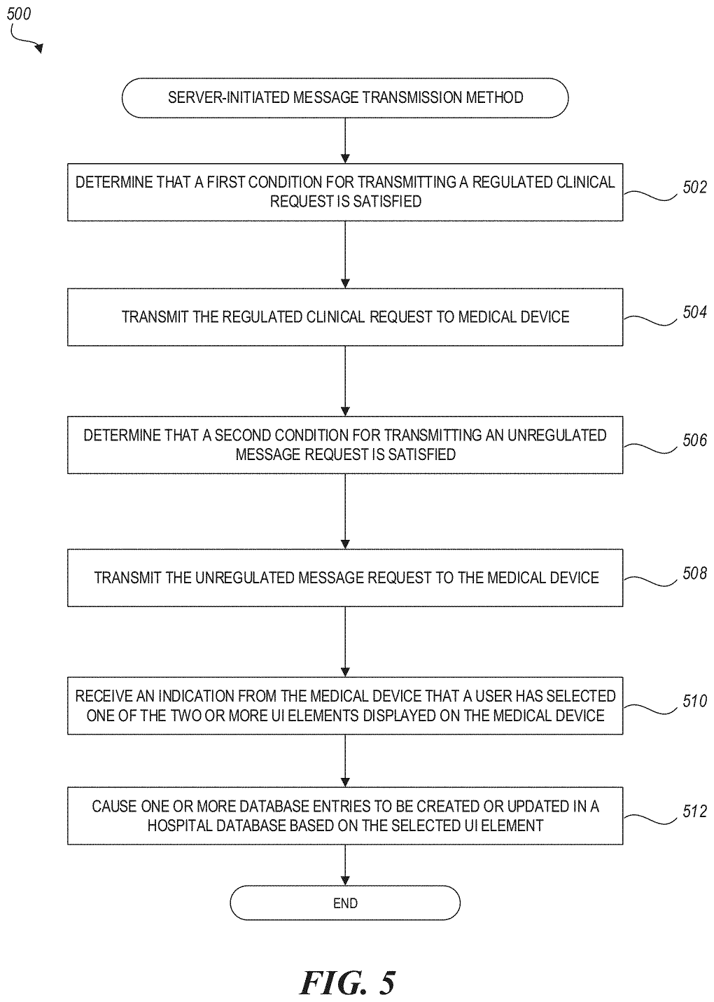

With reference now to FIG. 5, an example server-initiated message transmission method 500 will be described. The example method 500 may be carried out, for example, by the MMU server 3108 of FIG. 1 or the hospital server 206A of FIG. 2 (or one or more components thereof). For convenience, the steps of the example method 500 are described as being performed by a server. The method 500 illustrates an example algorithm that may be programmed, using any suitable programming environment or language, to create machine code capable of execution by a CPU or microcontroller of the server. Various embodiments may be coded using assembly, C, OBJECTIVE-C, C++, JAVA, or other human-readable languages and then compiled, assembled, or otherwise transformed into machine code that can be loaded into read-only memory (ROM), erasable programmable read-only memory (EPROM), or other recordable memory of the server that is coupled to the CPU or microcontroller and then then executed by the CPU or microcontroller.

At block 502, the server determines that a first condition for transmitting a regulated clinical request to the medical device is satisfied. For example, the first condition may be detecting an order for an infusion therapy based on the one or more parameters, where the one or more parameters indicate infusion parameters such as a medication to be infused and a concentration at which the medication is to be infused. Additionally, the regulated clinical request may be a type of communication to the medical device that is regulated by a governing entity. For example, the regulated clinical request may be an instruction to perform a first clinical operation (e.g., initiate an infusion therapy) according to one or more parameters (e.g., provided in the regulated clinical request or otherwise retrieved by the medical device).

At block 504, the server transmits the regulated clinical request to the medical device to cause the first clinical operation to be performed by the medical device according to the one or more parameters. In response to receiving the regulated clinical request, the medical device may perform the first clinical operation according to the one or more parameters. For example, the medical device may initiate an infusion therapy according to the infusion parameters included in the regulated clinical request.

At block 506, the server determines that a second condition for transmitting an unregulated message request to the medical device is satisfied. For example, the second condition may include one or more of (i) detecting that the medical device is within a threshold level of proximity with a patient not associated with the medical device, (ii) detecting that an ongoing infusion therapy at the medical device has been ordered to be discontinued, (iii) detecting that the medical device has entered a clinical care area not associated with the medical device, (iv) detecting that a medication bag associated with an ongoing infusion therapy at the medical device has reached a threshold level of volume, and (v) detecting that the medical device needs to be moved to a specific location. Additionally, the unregulated message request may be a type of communication is not regulated by the governing entity (e.g., due to the nature of the communication not being related to the clinical operations of the medical device). The unregulated message request may be an instruction to cause one or more indications to be output via the output devices of the medical device. For example, the one or more indications can include one or more visual or voice/audio messages to be output via the output devices of the medical device, and/or one or more user interface elements to be displayed on the display of the medical device.

At block 508, the server transmits the unregulated message request to the medical device to cause the one or more indications to be output via the output devices of the medical device. The server may generate a message request including the parameters that can be used to output the message on the medical device and transmit the message request to the medical device. For example, the message request may include data indicative of the content of the message to be output (e.g., “Would you like to associated this infusion pump with CCA X?” and/or other voice messages, alerts, and sounds), data indicative of the options to be presented to the user at the infusion pump (e.g., as selectable user interface elements) including the content associated with the options (e.g., “Yes,” “No,” etc.) and the parameters associated with the options (e.g., option identifiers, other identifiers associated with the options such as patient identifiers, CCA identifiers, etc. to be associated with the medical device upon user selection of the corresponding user interface elements). In response, the medical device may output the one or more indications via the output devices of the medical device using the data included or associated with the received message request. For example, the one or more indications may include two user interface elements that, upon user selection, provides a corresponding indication to the hospital server.

At block 510, the server receives an indication from the medical device that a user at the medical device has selected, via the one or more input devices of the medical device, a user interface element that was outputted on the medical device in response to the unregulated message request.

At block 512, the server causes one or more database entries to be created or updated in a hospital database based on the selected user interface element. For example, in the event that the unregulated message request requested confirmation from the user at the medical device that the medical device is to be associated with a given clinical care area or a given patient, the hospital server may update the hospital database to include such association(s) indicated by the user selection made at the medical device.

In the method 500, one or more of the blocks shown in FIG. 5 may be removed (e.g., not performed) and/or the order in which the method 500 is performed may be switched. In some embodiments, additional blocks may be added to the method 500. The embodiments of the present disclosure are not limited to or by the example shown in FIG. 5, and other variations may be implemented without departing from the spirit of this disclosure.

### Example Embodiments

In one embodiment, a system configured to facilitate transmission of messages between one or more medical devices and one or more hospital servers includes: a medical device and a hospital server. The medical device is configured to perform one or more clinical operations, wherein the medical device includes a display and one or more input devices. The hospital server is configured to transmit instructions to perform one or more of the clinical operations to the medical device, and transmit instructions to display one or more user interface elements on the display of the medical device. The hospital server is further configured to: determine that a first condition for transmitting a regulated clinical request to the medical device is satisfied, wherein the regulated clinical request comprises a type of communication to the medical device that is regulated by a governing entity and includes an instruction to perform a first clinical operation according to one or more parameters; transmit the regulated clinical request to the medical device to cause the first clinical operation to be performed by the medical device according to the one or more parameters; determine that a second condition for transmitting an unregulated message request to the medical device is satisfied, wherein the unregulated message request is a type of communication is not regulated by the governing entity and includes an instruction to cause two or more user interface elements to be displayed on the display of the medical device; transmit the unregulated message request to the medical device to cause the two or more user interface elements to be displayed on the display of the medical device; receive an indication from the medical device that a user at the medical device has selected one of the two or more user interface elements via the one or more input devices of the medical device; and cause one or more database entries to be created or updated in a hospital database based on the selected one of the two or more user interface elements.

In one embodiment, determining that the first condition is satisfied comprises detecting an order for an infusion therapy based on the one or more parameters, wherein the one or more parameters indicate at least a medication to be infused and a concentration at which the medication is to be infused. In one embodiment, determining that the second condition is satisfied comprises detecting that the medical device is within a threshold level of proximity with a patient not associated with the medical device, wherein the two or more user interface elements comprise a user interface element that, when selected by the user at the medical device, indicates to the hospital server that the patient is to be associated with the medical device, wherein the hospital server is further configured to update the hospital database to indicate that the patient is associated with the medical device.

In one embodiment, the medical device, in response to the selection by the user at the medical device, is configured to transmit the indication to the hospital server without changing one or more clinical operations being performed by the medical device. In one embodiment, determining that the second condition is satisfied comprises detecting that the medical device has entered a clinical care area not associated with the medical device, wherein the two or more user interface elements comprise a user interface element that, when selected by the user at the medical device, indicates to the hospital server that the medical device is to be associated with the clinical care area, wherein the hospital server is further configured to update the hospital database to indicate that the medical device is associated with the clinical care area.

In one embodiment, a server is configured to transmit messages to a medical device over a hospital network. The server is configured to: determine that a first condition for transmitting a clinical request to the medical device is satisfied, wherein the clinical request comprises an instruction to perform a first clinical operation according to one or more parameters; transmit the clinical request to the medical device to cause the first clinical operation to be performed by the medical device according to the one or more parameters; determine that a second condition for transmitting a message request to the medical device is satisfied, wherein the message request comprises an instruction to cause one or more indications to be outputted via one or more output devices of the medical device; and transmit the message request to the medical device to cause the one or more indications to be outputted via the one or more output devices of the medical device.

In one embodiment, determining that the first condition is satisfied comprises detecting an order for an infusion therapy based on the one or more parameters, wherein the one or more parameters indicate at least a medication to be infused and a concentration at which the medication is to be infused. In one embodiment, determining that the second condition is satisfied comprises detecting that the medical device is within a threshold level of proximity with a patient not associated with the medical device, wherein the one or more indications comprise a user interface element that, when selected by the user at the medical device, indicates to the server that the patient is to be associated with the medical device, wherein the server is further configured to update a hospital database to indicate that the patient is associated with the medical device.

In one embodiment, the medical device, in response to the selection by the user at the medical device, is configured to transmit, without changing one or more clinical operations being performed by the medical device, an indication to the server that the patient is to be associated with the medical device. In one embodiment, determining that the second condition is satisfied comprises detecting that an ongoing infusion therapy at the medical device has been ordered to be discontinued, wherein the one or more indications comprise a user interface element that indicates that the ongoing infusion therapy has been discontinued.

In one embodiment, the medical device is configured to output the user interface element that indicates that the ongoing infusion therapy has been discontinued on a display of the medical device without discontinuing the ongoing infusion therapy. In one embodiment, determining that the second condition is satisfied comprises detecting that the medical device has entered a clinical care area not associated with the medical device, wherein the one or more indications comprise a user interface element that, when selected by the user at the medical device, indicates to the server that the medical device is to be associated with the clinical care area, wherein the server is further configured to update a hospital database to indicate that the medical device is associated with the clinical care area.

In one embodiment, determining that the second condition is satisfied comprises detecting that a medication bag associated with an ongoing infusion therapy at the medical device has reached a threshold level of volume, wherein the one or more indications comprise a user interface element that indicates a location of another bag associated with the ongoing infusion therapy. In one embodiment, determining that the second condition is satisfied comprises detecting that the medical device needs to be moved to a specific location, wherein the one or more indications comprise a user interface element that provides an instruction to move the medical device to the specific location.

In one embodiment, a method of transmitting messages to a medical device over a hospital network, comprises: determining that a first condition for transmitting a clinical request to the medical device is satisfied, wherein the clinical request comprises an instruction to perform a first clinical operation according to one or more parameters; transmitting the clinical request to the medical device to cause the first clinical operation to be performed by the medical device according to the one or more parameters; determining that a second condition for transmitting a message request to the medical device is satisfied, wherein the message request comprises an instruction to cause one or more indications to be outputted via one or more output devices of the medical device; and transmitting the message request to the medical device to cause the one or more indications to be outputted via the one or more output devices of the medical device.

In one embodiment, determining that the first condition is satisfied comprises detecting an order for an infusion therapy based on the one or more parameters, wherein the one or more parameters indicate at least a medication to be infused and a concentration at which the medication is to be infused. In one embodiment, determining that the second condition is satisfied comprises detecting that the medical device is within a threshold level of proximity with a patient not associated with the medical device, wherein the one or more indications comprise a user interface element that, when selected by the user at the medical device, indicates to the server that the patient is to be associated with the medical device, wherein the server is further configured to update a hospital database to indicate that the patient is associated with the medical device.

In one embodiment, the medical device, in response to the selection by the user at the medical device, is configured to transmit, without changing one or more clinical operations being performed by the medical device, an indication to the server that the patient is to be associated with the medical device. In one embodiment, determining that the second condition is satisfied comprises detecting that an ongoing infusion therapy at the medical device has been ordered to be discontinued, wherein the one or more indications comprise a user interface element that indicates that the ongoing infusion therapy has been discontinued. In one embodiment, the medical device is configured to output the user interface element that indicates that the ongoing infusion therapy has been discontinued on a display of the medical device without discontinuing the ongoing infusion therapy.

### Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A system configured to facilitate transmission of messages between one or more medical devices and one or more hospital servers, the system comprising:
a medical device configured to perform one or more clinical operations, wherein the medical device includes a display and one or more input devices;
a hospital server configured to transmit instructions to perform one or more of the clinical operations to the medical device, and transmit instructions to display one or more user interface elements on the display of the medical device,
wherein the hospital server is further configured to:
determine that a first condition for transmitting a regulated clinical request to the medical device is satisfied, wherein the regulated clinical request comprises a type of communication to the medical device that is regulated by a governing entity and includes an instruction to perform a first clinical operation according to one or more parameters;
transmit the regulated clinical request to the medical device to cause the first clinical operation to be performed by the medical device according to the one or more parameters;
determine that a second condition for transmitting an unregulated message request to the medical device is satisfied, wherein the unregulated message request is a type of communication is not regulated by the governing entity and includes an instruction to cause two or more user interface elements to be displayed on the display of the medical device;
transmit the unregulated message request to the medical device to cause the two or more user interface elements to be displayed on the display of the medical device;
receive an indication from the medical device that a user at the medical device has selected one of the two or more user interface elements via the one or more input devices of the medical device; and
cause one or more database entries to be created or updated in a hospital database based on the selected one of the two or more user interface elements.

2. The system of claim 1, wherein determining that the first condition is satisfied comprises detecting an order for an infusion therapy based on the one or more parameters, wherein the one or more parameters indicate at least a medication to be infused and a concentration at which the medication is to be infused.

3. The system of claim 1, wherein determining that the second condition is satisfied comprises detecting that the medical device is within a threshold level of proximity with a patient not associated with the medical device, wherein the two or more user interface elements comprise a user interface element that, when selected by the user at the medical device, indicates to the hospital server that the patient is to be associated with the medical device, wherein the hospital server is further configured to update the hospital database to indicate that the patient is associated with the medical device.

4. The system of claim 3, wherein the medical device, in response to the selection by the user at the medical device, is configured to transmit the indication to the hospital server without changing one or more clinical operations being performed by the medical device.

5. The system of claim 1, wherein determining that the second condition is satisfied comprises detecting that the medical device has entered a clinical care area not associated with the medical device, wherein the two or more user interface elements comprise a user interface element that, when selected by the user at the medical device, indicates to the hospital server that the medical device is to be associated with the clinical care area, wherein the hospital server is further configured to update the hospital database to indicate that the medical device is associated with the clinical care area.

6. A server configured to transmit messages to a medical device over a hospital network, the server configured to:
determine that a first condition for transmitting a clinical request to the medical device is satisfied, wherein the clinical request comprises an instruction to perform a first clinical operation according to one or more parameters;
transmit the clinical request to the medical device to cause the first clinical operation to be performed by the medical device according to the one or more parameters;
determine that a second condition for transmitting a message request to the medical device is satisfied, wherein the message request comprises an instruction to cause one or more indications to be outputted via one or more output devices of the medical device; and
transmit the message request to the medical device to cause the one or more indications to be outputted via the one or more output devices of the medical device.

7. The server of claim 6, wherein determining that the first condition is satisfied comprises detecting an order for an infusion therapy based on the one or more parameters, wherein the one or more parameters indicate at least a medication to be infused and a concentration at which the medication is to be infused.

8. The server of claim 6, wherein determining that the second condition is satisfied comprises detecting that the medical device is within a threshold level of proximity with a patient not associated with the medical device, wherein the one or more indications comprise a user interface element that, when selected by the user at the medical device, indicates to the server that the patient is to be associated with the medical device, wherein the server is further configured to update a hospital database to indicate that the patient is associated with the medical device.

9. The server of claim 8, wherein the medical device, in response to the selection by the user at the medical device, is configured to transmit, without changing one or more clinical operations being performed by the medical device, an indication to the server that the patient is to be associated with the medical device.

10. The server of claim 6, wherein determining that the second condition is satisfied comprises detecting that an ongoing infusion therapy at the medical device has been ordered to be discontinued, wherein the one or more indications comprise a user interface element that indicates that the ongoing infusion therapy has been discontinued.

11. The server of claim 10, wherein the medical device is configured to output the user interface element that indicates that the ongoing infusion therapy has been discontinued on a display of the medical device without discontinuing the ongoing infusion therapy.

12. The server of claim 6, wherein determining that the second condition is satisfied comprises detecting that the medical device has entered a clinical care area not associated with the medical device, wherein the one or more indications comprise a user interface element that, when selected by the user at the medical device, indicates to the server that the medical device is to be associated with the clinical care area, wherein the server is further configured to update a hospital database to indicate that the medical device is associated with the clinical care area.

13. The server of claim 6, wherein determining that the second condition is satisfied comprises detecting that a medication bag associated with an ongoing infusion therapy at the medical device has reached a threshold level of volume, wherein the one or more indications comprise a user interface element that indicates a location of another bag associated with the ongoing infusion therapy.

14. The server of claim 6, wherein determining that the second condition is satisfied comprises detecting that the medical device needs to be moved to a specific location, wherein the one or more indications comprise a user interface element that provides an instruction to move the medical device to the specific location.

15. A method of transmitting messages to a medical device over a hospital network, the method comprising:
determining that a first condition for transmitting a clinical request to the medical device is satisfied, wherein the clinical request comprises an instruction to perform a first clinical operation according to one or more parameters;
transmitting the clinical request to the medical device to cause the first clinical operation to be performed by the medical device according to the one or more parameters;
determining that a second condition for transmitting a message request to the medical device is satisfied, wherein the message request comprises an instruction to cause one or more indications to be outputted via one or more output devices of the medical device; and
transmitting the message request to the medical device to cause the one or more indications to be outputted via the one or more output devices of the medical device.

16. The method of claim 15, wherein determining that the first condition is satisfied comprises detecting an order for an infusion therapy based on the one or more parameters, wherein the one or more parameters indicate at least a medication to be infused and a concentration at which the medication is to be infused.

17. The method of claim 15, wherein determining that the second condition is satisfied comprises detecting that the medical device is within a threshold level of proximity with a patient not associated with the medical device, wherein the one or more indications comprise a user interface element that, when selected by the user at the medical device, indicates to the server that the patient is to be associated with the medical device, wherein the server is further configured to update a hospital database to indicate that the patient is associated with the medical device.

18. The method of claim 17, wherein the medical device, in response to the selection by the user at the medical device, is configured to transmit, without changing one or more clinical operations being performed by the medical device, an indication to the server that the patient is to be associated with the medical device.

19. The method of claim 15, wherein determining that the second condition is satisfied comprises detecting that an ongoing infusion therapy at the medical device has been ordered to be discontinued, wherein the one or more indications comprise a user interface element that indicates that the ongoing infusion therapy has been discontinued.

20. The method of claim 19, wherein the medical device is configured to output the user interface element that indicates that the ongoing infusion therapy has been discontinued on a display of the medical device without discontinuing the ongoing infusion therapy.
